(19) United States
(12) Patent Application Publication
(10) Pub. No.: US 2007/0231802 A1
(43) Pub. Date: Oct. 4, 2007

(54) Method for evaluating integrity of a genomic sample

(76) Inventor: Michael T. Barrett, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/394,562

(22) Filed: Mar. 31, 2006

(51) Int. Cl. C12Q 1/68; C12M 3/00

(52) U.S. Cl. 435/6; 435/287.2

(57) ABSTRACT

Described herein is a method of evaluating a genomic sample. One embodiment of the instant method generally includes amplifying a relatively long genomic sequence and a relatively short genomic sequence from a genomic sample, and comparing the amounts of amplification products produced.

### BACKGROUND

Many genomic and genetic studies are directed to the identification of differences in gene dosage or expression among cell populations for the study and detection of disease. For example, many malignancies involve the gain or loss of DNA sequences resulting in activation of oncogenes or inactivation of tumor suppressor genes. Identification of the genetic events leading to neoplastic transformation and subsequent progression can facilitate efforts to define the biological basis for disease, develop predictors of disease outcomes, improve prognosis of therapeutic response, and permit earlier tumor detection. In addition, perinatal genetic problems frequently result from loss or gain of chromosome segments such as trisomy 21 or the deletion syndromes. Methods of pre and postnatal detection of such abnormalities can be helpful in early diagnosis of disease.

Comparative genomic hybridization (CGH) is one approach that has been employed to detect the presence and identify the location of amplified or deleted sequences. In one implementation of CGH, genomic DNA is isolated from reference cells (e.g., normal cells), as well as from test cells (e.g., tumor cells). The two nucleic acids are differentially labeled and then simultaneously hybridized in situ to metaphase chromosomes of a reference cell. Chromosomal regions in the test cells which are at increased or decreased copy number relative to the reference cell can be identified by detecting regions where the ratio of the signals from the two distinguishably labeled nucleic acids is altered. For example, those regions that are at a lower copy number in the test cells show relatively lower signal from the test nucleic acids than the reference compared to other regions of the genome. Regions that are at a higher copy number in the test cells show relatively higher signal from the test nucleic acid.

In a recent variation of the above traditional CGH approach, the immobilized chromosome elements have been replaced with a collection of solid support surface-bound polynucleotides, e.g., an array of BAC (bacterial artificial chromosome) clones, cDNAs or oligonucleotides. Such array-based approaches offer benefits over immobilized chromosome approaches, including a higher resolution, as defined by the ability of the assay to localize chromosomal alterations to specific areas of the genome.

In general terms, the quality of the results obtained from a CGH assay (i.e., the degree of correspondence between the actual copy number of a genomic locus and the prediction made about the copy number of that genomic locus using data obtained from a CGH assay) largely depends on the quality of the genomic DNA sample used to perform the assay. Since the quality of a genomic DNA sample employed in a CGH assay may vary greatly (particularly in the case of genomic DNA samples obtained in a clinical setting), the quality of results obtained from a CGH assay may also vary greatly. For example, in certain cases, the genomic DNA in a sample employed in a CGH assay may be partially or completely degraded, which may make that genomic DNA difficult to effectively amplify and/or label.

### SUMMARY

Described herein is a method of evaluating a genomic sample. One embodiment of the method includes amplifying short and long nucleic acid sequences from a genomic sample to produce low and high molecular weight amplification products, and comparing the amounts of the produced amplification products. Also provided are protocols employing the method, as well as kits for performing the method.

### BRIEF DESCRIPTION OF THE DRAWINGS

FIG. 1 schematically illustrates exemplary results illustrating one aspect of one embodiment of the invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “nucleic acid” and “polynucleotide” are used interchangeably herein to describe a polymer of any length, e.g., greater than about 10 bases, greater than about 100 bases, greater than about 500 bases, greater than 1000 bases, up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from 2 to about 200 nucleotides in length, e.g., about 10 to 100 nucleotides in length. Oligonucleotides may be synthetic and, in many embodiments, are about 20 to about 60 nucleotides in length.

The term “sample” as used herein relates to a material or mixture of materials, typically, although not necessarily, in fluid form, containing one or more components (i.e., analytes) of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, oligonucleotides may be present on a planar surface of a support, e.g., in the form of an array.

The phrase “labeled population of nucleic acids” refers to a mixture of nucleic acids that is detectably labeled, e.g., fluorescently labeled, such that the presence of the nucleic acids can be detected by assessing the presence of the label. If a labeled population of nucleic acids is made from or made using a genomic sample, the sample is usually employed as template for making the population of nucleic acids.

The term “array” encompasses the term “microarray” and refers to an ordered array presented for binding to nucleic acids and the like.

An “array,” includes any two-dimensional or substantially two-dimensional (as well as a three-dimensional) arrangement of spatially addressable regions bearing nucleic acids, particularly oligonucleotides or synthetic mimetics thereof, and the like, e.g., UNA oligonucleotides. Where the arrays are arrays of nucleic acids, the nucleic acids may be adsorbed, physisorbed, chemisorbed, or covalently attached to the arrays at any point or points along the nucleic acid chain.

The term “substrate” as used herein refers to a surface upon which marker molecules or probes, e.g., an array, may be adhered. Substrates may be porous or non-porous, planar or non-planar over all or a portion of their surface. Glass slides are the most common substrate for arrays, although fused silica, silicon, plastic and other materials are also suitable.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 mm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” or “sample composition” and the like will be referenced as a moiety in a mobile phase (typically fluid), to be detected by “surface-bound polynucleotides” which are bound to the substrate at the various regions. These phrases are synonymous with the arbitrary terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equilvalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent hybridization conditions may also include a “prehybridization” of aqueous phase nucleic acids with complexity-reducing nucleic acids to suppress repetitive sequences and reduce the complexity of the sample prior to hybridization. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of surface-bound polynucleotides, as is commonly known in the art and described below, is not a mixture of surface-bound polynucleotides because the species of surface-bound polynucleotides are spatially distinct and the array is addressable. “Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION

Described herein is a method of evaluating a genomic sample. One embodiment of the method includes amplifying short and long nucleic acid sequences from a genomic sample to produce low and high molecular weight amplification products, and comparing the amounts of the produced amplification products. Also provided are protocols employing the method, as well as kits for performing the method.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Representative embodiments of the subject methods are described in greater detail below, followed by a description of representative protocols in which the subject methods find use. Finally, kits for performing the subject method are described.

Method of Sample Analysis

An exemplary embodiment of the method summarized above includes: a) amplifying a relatively short nucleic acid sequence from a genomic sample to produce an amount of a relatively low molecular weight amplification product; b) amplifying a relatively long nucleic acid sequence from the genomic sample to produce an amount of a relatively high molecular weight amplification product; and c) comparing the amount of the relatively low molecular weight amplification product to the amount of the high relatively molecular weight amplification product, to evaluate the genomic sample.

As will be discussed in greater detail below, the relative abundance of the high and low molecular weight amplification products provides an evaluation of the integrity of the genomic DNA of a genomic sample. In particular embodiments, the abundance of the high and low molecular weight amplification products obtained using a test genomic sample may be compared to produce a ratio. That ratio may, in certain embodiments, be compared to a reference ratio to provide an evaluation of the genomic sample. The reference ratio may be obtained using a control genomic sample, e.g., a genomic sample containing a genome of known integrity.

The genomic sample employed in the subject method generally contains genomic DNA or an amplified version thereof (e.g., genomic DNA amplified using the methods of Lage et al, Genome Res. 2003 13: 294-307 or published patent application US20040241658, for example) from the nuclei of eukaryotic cells. In exemplary embodiments, the genomic sample may contain genomic DNA from a mammalian cell such a human, mouse, rat or monkey cell. The cells used to produce a genomic sample may be cultured cells or cells of a clinical sample, e.g., a tissue biopsy, scrape or lavage and, in certain embodiments, may or may not be cells of a forensic sample (i.e., cells of a sample collected at a crime scene). In particular embodiments, the genomic sample may be derived (e.g., made from) from an archived sample (which may or may not be a cellular sample) that has been stored prior to use (e.g., stored prior to labeling or stored prior to extraction of genomic DNA from the sample). If employed, an archived sample may have been stored under any condition, e.g., at below room temperature (e.g., frozen such as at about −80° C., at about −20° C. or at about 4° C.), at room temperature (e.g., at about 20° C.), above room temperature, at below atmospheric pressure (e.g., in a vacuum), above atmospheric pressure (e.g., under pressure) or at atmospheric pressure (about 760 Torr) for several hours, days, weeks or years prior to use, for example. The genomic DNA content of a genomic sample may be undetermined (i.e., known or unknown), prior to performing the subject methods. Likewise, the integrity of the genomic DNA of a genomic sample may be undetermined prior to performing the subject methods. In particular embodiments, the genomic DNA of a genomic sample may be intact, i.e., substantially undegraded (e.g., containing genomic DNA that is less than about 10% degraded). In other embodiments, the genomic DNA of a genomic sample may be substantially degraded (i.e., containing genomic DNA that is at least about 10% degraded, e.g., at least about 50%, at least about 80%, at least about 90% or at least about 95% or about 99% degraded), where degradation of genomic DNA may be calculated by determining the amount of the genomic DNA that is below about 100 kb in length, relative to the amount of genomic DNA that is above about 100 kb in length. Although there is no requirement to know the amount of genomic DNA that is present in a genomic sample used in the subject method, genomic DNA at concentrations of about 0.1 pg/μl to about 1 pg/μl, about 1 pg/μl to about 10 pg/μl, 10 pg/μl to about 0.1 ng/μl, 0.1 ng/μl to about 1 ng/μl, about 1 ng/μl to about 10 ng/μl, about 10 ng/μl to about 100 ng/μl, about 100 ng/μl to about 1 μg/μl of genomic DNA are readily employed in the instant methods

The first steps of the subject method are generally similar to conventional array-based CGH assays in that a genomic sample is obtained by, for example, receiving a genomic sample or producing a genomic sample from cells. Methods for making such genomic samples are generally well known in the art and described in the publications discussed in the background section herein, and in well known laboratory manuals (e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y. for example).

After a genomic sample is obtained, the relative abundance of two different genomic sequences in the genomic sample is evaluated. One of the genomic sequences, arbitrarily referred to herein as a “first genomic sequence” is a relatively short genomic sequence whereas the other genomic sequence, arbitrarily referred to herein as a “second genomic sequence” is a relatively long genomic sequence. The relatively short genomic sequence is shorter than the relatively long genomic sequence. The first genomic sequence, in certain embodiments, is between about 50 nt and about 500 nt in length, e.g., about 100 to about 200 nt, about 200 to about 300 nt or about 400 to about 500 nt in length, whereas the second genomic sequence, in certain embodiments, is between about 2.0 kb to about 10.0 kb in length, e.g., about 2.0 kb to about 3.0 kb, about 3.0 kb to about 4.0 kb, about 4.0 kb to about 5.0 kb, about 5.0 kb to about 5.0 kb or about 5.0 kb to about 10.0 kb in length, or greater. In certain embodiments, the second genomic sequence may be at least about 5 times, at least about 10 times, at least about 20 times or at least about 50 times longer than the length of the first genomic sequence.

In addition to being of differing lengths, the first and second genomic sequences may be present as single copy number sequences in the genome of the genomic sample (i.e., occurring as a single copy in a single position of the genome). In other embodiments, the first and second genomic sequences may be each present in multiple copies in the genome of the genomic sample. If the first and second genomic sequences are present in multiple copies in the genome, those sequences may, in particular embodiments, have a copy number of at least about 10, at least 100, at least 1,000, at least 10,000 or at least 100,000, in the genome. In certain embodiments, the multiple genomic sequences may be distributed throughout the genome (i.e., may be interspersed, evenly or unevenly, throughout the genome). The first and second genomic sequences may be arbitrarily or empirically chosen and may be any suitable regions of a genome.

In certain embodiments, the first and second genomic sequences may be repetitive elements that are interspersed throughout the genome under investigation. Exemplary repetitive elements that are suitable for use in the subject method include long interspersed repeated sequences (LINES), short interspersed repeated sequences (SINES), and other transposable element-derived repeated elements such as LTR elements, DNA transposon elements and pseudogenes. The first genomic sequence (i.e., the relatively short sequence) may be a sequence of any repetitive element. In certain embodiments, the first genomic sequence may be a SINE such as an Alu (e.g., Alu1, Alu2 or Alu3) or MIR (mammalian interspersed region), for example. The second genomic sequence (i.e., the relatively long sequence) may be a sequence a LINE, such as a L1 or L2 element, for example. Representative repetitive elements that may be employed in the instant methods are described in a variety of publications, including Weiner et al., “SINEs and LINEs: the art of binding the hand that feeds you” Curr. Opin. Cell Biol. (2002) 14: 343-350; Smit et al., “Interspersed repeats and other mementos of transposable elements in mammalian genomes” Curr. Opin. Genet. Dev. (1999) 657-663; Ovchinnikov et al., “Tracing the LINEs of human evolution” Proc. Natl. Acad. Sci. (2002) 99:10522-10527; Scheen et al., “Reading between the LINEs: human genomic variation induced by LINE-1 retrotransposition” Genome Res. (2000) 10: 1496-1508 and Deininger et al., “Mammalian retroelements” Genome Research (2002) 12:1455-1465.

As mentioned above and in certain embodiments, the first and second genomic sequences may be amplified from a single genomic sample to produce a relatively low molecular weight amplification product and a relatively high molecular weight amplification product, respectively. The high and low molecular weight amplification products are generally produced by contacting the genomic sample with suitable primers for amplifying the genomic sequences and a polymerase (e.g., a thermostable polymerase), and maintaining the genomic sample, primers and polymerase under conditions suitable for amplifying the genomic sequences. In certain embodiments, the high and low molecular weight amplification products may be produced by polymerase chain reaction, the conditions for which reactions are well known in the art. The first and second genomic sequences may be amplified in the same or in different reactions.

If polymerase chain reaction is employed to produce amplification products from the first and second genomic sequences, the amounts of the amplification products may be assessed during a stage at which the nucleic acid amplification occurs linearly (i.e., during the linear phase of the amplification reactions). In certain cases, the reaction may be terminated at that stage. In certain embodiments therefore, if polymerase chain reaction is employed, less than about 12, e.g., 3, 4,5, 6, 7, 8, 9, 10 or 11 rounds of amplification (e.g., successive cycles of denaturation, re-naturation and polymerization) may be employed in the reaction. In general, the number of rounds of application employed provides an amount of amplification product that is detectable using the detection system employed. The optimal number of rounds of amplification employed in the subject methods may vary from sample to sample, the genomic sequences chosen for amplification, and the method used for detection of the amplification products. The optimal number of rounds of amplification for each genomic sample is readily determinable. In one embodiment (described in greater detail below) “real time” amplification methods may be employed in which the amount of amplification products produced in a reaction may be monitored without terminating the reaction.

Since, as would be apparent from the preceding description, the nucleotide sequence of first and second genomic sequences may vary greatly, the nucleotide sequences of the primers used to amplify the genomic sequences may, also, vary greatly. However, since the genomes of many eukaryotic organisms have been sequenced and those sequences have been annotated and deposited into public databases such as NCBI's Genbank Database, the primers that could be used in the instant methods are readily designed. Exemplary primers suitable for use in amplifying SINE and LINE repeats are described in a variety of publications, including: Lichter et al., Proc. Natl. Acad. Sci. (1990) 87:6634-8; Lengauer et al., Genomics (1992) 13:826-8; Nicklas et al., J. Forensic Sci. (2003) 48:936-44, Walker et al., Anal. Biochem. (2003) 315:122-8, Tringali et al., Forensic Sci. Int. (2004) 146 Suppl:S177-81, Ovchinnikov et al., Proc. Nat'l. Acad. Sci. USA (2002) 99:10522-10527 and Boissonot et al., Molecular Biology and Evolution (2000) 17:915-928. In certain embodiments, detectably labeled (e.g., fluorescent) primers may be employed.

After amplification, the amount of the high molecular weight amplification products and the amount of the low molecular weight amplification products may be assessed. The amount of amplification products may be assessed by any suitable means, including, but not limited to: separating the products according to their size using a separation device (for example, a column, gel or filter) and independently detecting the presence of the separated products by, e.g., a) contacting the separated products with a detectable (e.g., fluorescent) DNA binding agent and assessing the amount of bound agent, b) by detecting absorbance at 260 nm, or, c) detecting the presence of a detectable label if a detectably labeled primer was employed in the amplification reaction. In another embodiment, the amount of amplification products may be assessed using quantitative or so called “real-time” PCR methods that do not require separation of the amplification products. Real-time PCR methods, such as those described in Nicklas et al., J. Forensic Sci. (2005) 50:1081-90; Orlando et al., Clin. Chem. Lab. Med. (1998) 36:255-69; Nicklas et al., J. Forensic Sci. (2003) 48:936-44 and Schneider et al., Clin. Exp. Metastasis (2002) 19:571-82, are readily adapted for employment in the instant methods. The methods described above are readily automated. In certain embodiments, a microfluidic system may be employed for analysis of amplification products. One representative system that may be employed is the DNA 7500 LabChip and Bioanalyzer of Agilent Technologies (Palo Alto, Calif.).

The relatively low molecular weight amplification product, in certain embodiments, is about 50 nt to about 500 nt in length, e.g., about 100 to about 200 nt, about 200 to about 300 nt or about 400 to about 500 nt in length, whereas the relatively high molecular weight amplification product, in certain embodiments, is about 2.0 kb to about 10.0 kb in length, e.g., about 2.0 kb to about 3.0 kb, about 3.0 kb to about 4.0 kb, about 4.0 kb to about 5.0 kb, about 5.0 kb to about 5.0 kb or about 5.0 kb to about 10.0 kb in length, or greater. In a particular embodiment, the relatively low molecular weight amplification product is between about 100 nt and about 300 nt in length and the relatively high molecular weight amplification product is between about 3 kb to about 7 kb in length. The relatively high molecular weight amplification product may have a molecular weight that is at least about 5 times, at least about 10 times, at least about 20 times or at least about 50 times greater than the relatively molecular weight of the low molecular weight amplification product.

The amount of the relatively low molecular weight amplification product and the amount of the relatively high molecular weight amplification product may then be compared to provide a qualitative or quantitative evaluation of the genomic sample. In certain embodiments, the results of the comparison may be numerically expressed, e.g., as a ratio (as a number, fraction, integer, or the like) that represents the relative amounts of the low and high molecular weight amplification products produced.

In particular embodiments, the numerical expression that represents the relative amounts of the low and high molecular weight amplification products produced may be compared to a reference numerical expression (e.g., a reference ratio). The reference numerical expression may be arbitrarily or empirically chosen, and, in certain embodiments may be obtained using a control genomic sample. The control genomic sample, in certain embodiments, may contain genomic DNA of pre-determined (i.e., known) integrity, e.g., substantially undegraded (e.g., containing genomic DNA that is less than about 10% degraded) or substantially degraded. In certain embodiments the control genomic sample contains genomic DNA of a quality that is known to be suitable for use in an array-based CGH assay. For example, in certain embodiments, a ratio representing the relative abundance of amplification products produced from a test sample may be compared to a reference ratio that represents the relative abundance of the same amplification products (i.e., amplification products produced using the same primers as those used for amplification of the test sample) from a control sample. In certain embodiments, the control sample may be made from the same species, tissue type and/or cell-type as the test sample. As would be apparent to one of skill in the art, amplification reactions for test and control sample, if employed, may be performed in parallel or in series. Results obtained using a test sample may be compared to results obtained using a first control sample and a second control sample, where the first control sample may contain substantially undegraded genomic DNA and the second control sample may contain substantially degraded DNA.

In general terms, the closer a ratio from a test sample is to a reference ratio (e.g., a reference ratio obtained using a control sample containing intact genomic DNA), the more intact the genomic DNA of the test sample. In other words, if the ratio from a test sample is identical to or within 5%, 10%, 20% or, in certain embodiments, 30% of a reference ratio produced using a sample having an intact genome, the test sample may contain genomic DNA that is generally intact.

In addition to the above and in certain embodiments, the abundance of the relatively low molecular weight amplification product may be employed to evaluate the total amount of genomic DNA in a genomic sample, allowing for comparisons between different samples to be made. In one embodiment, any difference in concentration of genomic DNA in two genomic samples may be compensated for by using more or less of one of the samples.

The above-described protocols may be employed in a variety of methods, including in a) methods of identifying a test genomic sample suitable for use in an array-based comparative genome hybridization assay, b) methods of identifying a test genomic sample suitable for amplification, c) methods of identifying samples that amplified uniformly, and d) methods of selecting a test genomic sample. In general terms, these methods include comparing the above-referenced ratio to a reference ratio, and, based on this comparison, indicating whether a test sample is of a suitable quality for further use. Accordingly, the methods described above have a particular utility as a quality control step in providing samples of sufficient quality for use in, for example, array-based CGH experiments or amplification protocols.

Methods of identifying a test genomic sample suitable for use in an array-based comparative genome hybridization assay generally include: a) performing the instant methods on the test genomic sample to produce an assessment of the integrity of the test genomic sample and b) determining whether the assessment is above a threshold. In general, a test genomic sample having an assessment above a threshold indicates that the test genomic sample is suitable for use in an array-based comparative genome hybridization assay, or, in other methods, suitable for amplification.

Since many amplification methods (e.g., those described in Lage et al, Genome Res. 2003 13: 294-307 or published patent application US20040241658) require a relatively intact genome template for efficient amplification to occur, the instant methods may be readily employed to determine if a genomic sample is suitable for amplification. As would be readily apparent, if a genomic sample is deemed to have an integrity that is below a threshold integrity, that genomic sample may not be suitable for amplification. Likewise, if a genomic sample is deemed to have an integrity that is above a threshold integrity, the genomic sample may be suitable for amplification. In these methods, the integrity of a genomic sample may be tested using the above methods and, on the basis of the results obtained, the genomic sample may be deemed suitable or unsuitable for amplification. If the genomic sample is deemed suitable for amplification, it may be labeled and employed in a CGH assay described in greater detail below.

Methods of selecting a test genomic sample generally include: a) performing the instant methods on a plurality (e.g., 2 or more, e.g., 5 or more, 10 or more, 50 or more or 100 or more) of test genomic samples to produce a numerical assessment for each test sample; and b) selecting one or more test genomic samples from the plurality of test genomic samples based on whether the numerical assessment for each sample is above the threshold.

In certain embodiments of these methods, particularly if the amplification products are separated by size or other physical means, the degree of smearing or laddering of the amplification products may also be taken into consideration in deciding whether a test sample is of a suitable quality for further use.

Kits

Kits for use in accordance with the subject methods are also provided. The kits at least include a first primer pair for amplifying a relatively low molecular weight product from a first genomic sequence of a test genomic sample and a second primer pair for amplifying a relatively high molecular weight product from a second genomic sequence of the test genomic sample, as described above. A kit may include one or more of: a control genomic sample that contains a genome that is substantially undegraded or substantially degraded, reagents for labeling a genomic sample, a CGH array, and a device for size separation device for separating the high and low molecular weight amplification products. In one embodiment, the kit provides an integrated microfluidic device upon which the amplification products may be size separated and assessed

A subject kit may further include one or more additional components necessary for carrying out an array-based CGH assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

The kits may also include a computer readable medium including and instructions that may include directions for use of the invention.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

Utility

Samples evaluated, or, in certain embodiments, selected according to the above methods may be employed in array-based CGH binding assays. Such assays may be employed for the quantitative comparison of copy number of one nucleic acid sequence in a first collection of nucleic acid molecules relative to the copy number of the same sequence in a second collection.

The arrays employed in CGH assays contain polynucleotides immobilized on a solid support. Array platforms for performing the array-based methods are generally well known in the art (e.g., see Pinkel et al., Nat. Genet. (1998) 20:207-211; Hodgson et al., Nat. Genet. (2001) 29:459-464; Wilhelm et al., Cancer Res. (2002) 62: 957-960) and, as such, need not be described herein in any great detail. In general, CGH arrays contain a plurality (i.e., at least about 100, at least about 500, at least about 1000, at least about 2000, at least about 5000, at least about 10,000, at least about 20,000, usually up to about 100,000 or more) of addressable features that are linked to a planar solid support. Features on a subject array usually contain a polynucleotide that hybridizes with, i.e., binds to, genomic sequences from a cell. Accordingly, such “comparative genome hybridization arrays”, for short “CGH arrays” typically have a plurality of different BACs, cDNAs, oligonucleotides, or inserts from phage or plasmids, etc., that are addressably arrayed. As such, CGH arrays usually contain surface bound polynucleotides that are about 10-200 bases in length, about 201-5000 bases in length, about 5001-50,000 bases in length, or about 50,001-200,000 bases in length, depending on the platform used.

In particular embodiments, CGH arrays containing surface-bound oligonucleotides, i.e., oligonucleotides of 10 to 100 nucleotides and up to 200 nucleotides in length, find particular use in the subject methods.

In general, the subject assays involve labeling a test and a reference genomic sample to make two labeled populations of nucleic acids which may be distinguishably labeled, contacting the labeled populations of nucleic acids with an array of surface bound polynucleotides under specific hybridization conditions, and analyzing any data obtained from hybridization of the nucleic acids to the surface bound polynucleotides. Such methods are generally well known in the art (see, e.g., Pinkel et al., Nat. Genet. (1998) 20:207-211; Hodgson et al., Nat. Genet. (2001) 29:459-464; Wilhelm et al., Cancer Res. (2002) 62: 957-960)) and, as such, need not be described herein in any great detail.

Two different genomic samples may be differentially labeled, where the different genomic samples may include an “experimental” sample, i.e., a sample of interest, and a “control” sample to which the experimental sample may be compared. In certain embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell type may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells, or in different phases of the cell cycle) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

The genomic sample (containing intact, fragmented or enzymatically amplified chromosomes, or amplified fragments of the same), are distinguishably labeled using methods that are well known in the art (e.g., primer, extension, random-priming, nick translation, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). The samples are usually labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Bostan Mass.) and POPRO3 TOPRO3 (Molecular Probes, Eugene, Oreg.). Further suitable distinguishable detectable labels may be found in Kricka et al. (Ann Clin Biochem. 39:114-29, 2002).

The labeling reactions produce a first and second population of labeled nucleic acids that correspond to the test and reference chromosome compositions, respectively. After nucleic acid purification and any optional pre-hybridization steps to suppress repetitive sequences (e.g., hybridization with Cot-1 DNA), the populations of labeled nucleic acids are contacted to an array of surface bound polynucleotides, as discussed above, under conditions such that nucleic acid hybridization to the surface bound polynucleotides can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C.

The labeled nucleic acids can be contacted to the surface bound polynucleotides serially, or, in other embodiments, simultaneously (i.e., the labeled nucleic acids are mixed prior to their contacting with the surface-bound polynucleotides). Depending on how the nucleic acid populations are labeled (e.g., if they are distinguishably or indistinguishably labeled), the populations may be contacted with the same array or different arrays. Where the populations are contacted with different arrays, the different arrays are substantially, if not completely, identical to each other in terms of target feature content and organization.

Standard hybridization techniques (using high stringency hybridization conditions) are used to probe a target nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos: 6,335,167; 6,197,501; 5,830,645; and 5,665,549; the disclosures of which are herein incorporate by reference.

Generally, comparative genome hybridization methods comprise the following major steps: (1) immobilization of polynucleotides on a solid support; (2) pre-hybridization treatment to increase accessibility of support-bound polynucleotides and to reduce nonspecific binding; (3) hybridization of a mixture of labeled nucleic acids to the surface-bound nucleic acids, typically under high stringency conditions; (4) post-hybridization washes to remove nucleic acid fragments not bound to the solid support polynucleotides; and (5) detection of the hybridized labeled nucleic acids. The reagents used in each of these steps and their conditions for use vary depending on the particular application.

As indicated above, hybridization is carried out under suitable hybridization conditions, which may vary in stringency as desired. In certain embodiments, highly stringent hybridization conditions may be employed. The term “high stringent hybridization conditions” as used herein refers to conditions that are compatible to produce nucleic acid binding complexes on an array surface between complementary binding members, i.e., between the surface-bound polynucleotides and complementary labeled nucleic acids in a sample. Representative high stringency assay conditions that may be employed in these embodiments are provided above.

The above hybridization step may include agitation of the immobilized polynucleotides and the sample of labeled nucleic acids, where the agitation may be accomplished using any convenient protocol, e.g., shaking, rotating, spinning, and the like.

Following hybridization, the array-surface bound polynucleotides are typically washed to remove unbound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is read. Reading of the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications: Ser. No. 09/846125 “Reading Multi-Featured Arrays” by Dorsel et al.; and U.S. Pat. No. 6,406,849, which references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, the subject methods include a step of transmitting data or results from at least one of the detecting and deriving steps, also referred to herein as evaluating, as described above, to a remote location. By “remote location” is meant a location other than the location at which the array is present and hybridization occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart.

“Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Accordingly, a pair of chromosome compositions is labeled to make two populations of labeled nucleic acids, the nucleic acids contacted with an array of surface-bound polynucleotides, and the level of labeled nucleic acids bound to each surface-bound polynucleotide is assessed.

In certain embodiments, a surface-bound polynucleotide is assessed by determining the level of binding of the population of labeled nucleic acids to that polynucleotide. The term “level of binding” means any assessment of binding (e.g. a quantitative or qualitative, relative or absolute assessment) usually done, as is known in the art, by detecting signal (i.e., pixel brightness) from the label associated with the labeled nucleic acids. Since the level of binding of labeled nucleic acid to a surface-bound polynucleotide is proportional to the level of bound label, the level of binding of labeled nucleic acid is usually determined by assessing the amount of label associated with the surface-bound polynucleotide.

In certain embodiments, a surface-bound polynucleotide may be assessed by evaluating its binding to two populations of nucleic acids that are distinguishably labeled. In these embodiments, for a single surface-bound polynucleotide of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

By “normalization” is meant that data corresponding to the two populations of nucleic acids are globally normalized to each other, and/or normalized to data obtained from controls (e.g., internal controls produce data that are predicted to equal in value in all of the data groups). Normalization generally involves multiplying each numerical value for one data group by a value that allows the direct comparison of those amounts to amounts in a second data group. Several normalization strategies have been described (Quackenbush et al, Nat Genet. 32 Suppl:496-501, 2002, Bilban et al Curr Issues Mol Biol. 4:57-64, 2002, Finkelstein et al, Plant Mol Biol.48(1-2):119-31, 2002, and Hegde et al, Biotechniques. 29:548-554, 2000). Specific examples of normalization suitable for use in the subject methods include linear normalization methods, non-linear normalization methods, e.g., using lowess local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using the data obtained from a support-bound polynucleotide for a chromosome of known concentration in any of the chromosome compositions.

Accordingly, binding of a surface-bound polynucleotide to a labeled population of nucleic acids may be assessed. In most embodiments, the assessment provides a numerical assessment of binding, and that numeral may correspond to an absolute level of binding, a relative level of binding, or a qualitative (e.g., presence or absence) or a quantitative level of binding. Accordingly, a binding assessment may be expressed as a ratio, whole number, or any fraction thereof.

CGH assays may be used to identify abnormal nucleic acid copy number and mapping or investigating of chromosomal abnormalities associated with disease, e.g., cancer for example.

### EXAMPLE 1

Aliquots of amplified or extracted material are used as template in locus-specific PCR reactions. 1-10 ng of DNA is used per PCR reaction.

Two sets of primer are used (e.g., to amplify Alu1 and L1) either separately or in a duplex reaction. After a minimal number of cycles the samples are analyzed by gel electrophoresis or using another device such as an Agilent DNA 7500 LabChip and Bioanalyzer.

After separation the bands are quantified and used to derive both the total amount of template in each sample as well as its quality.

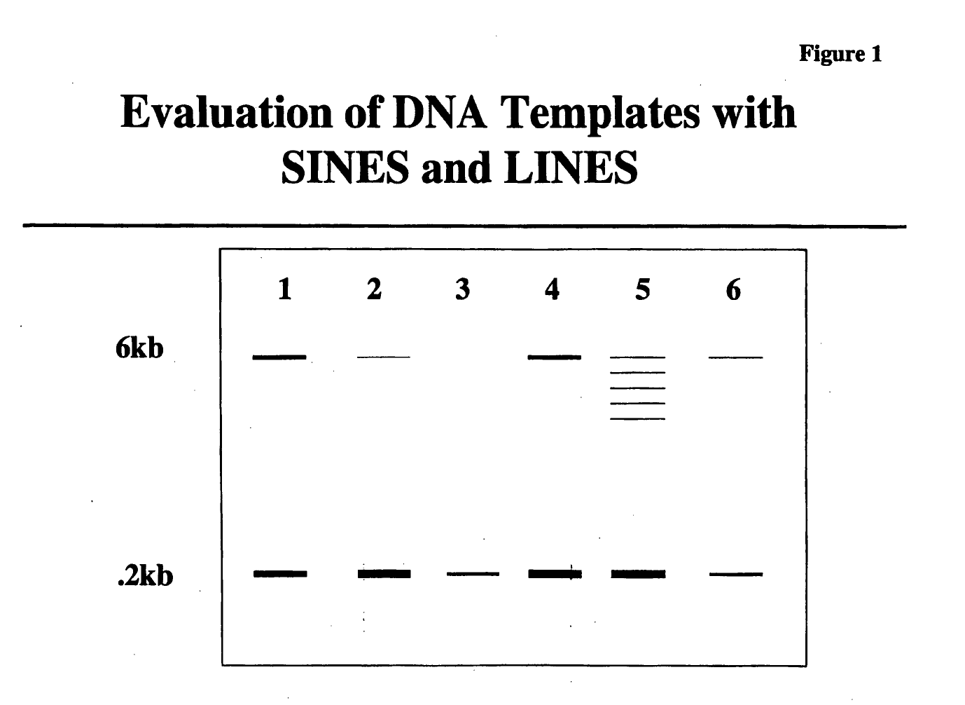

FIG. 1 illustrates hypothetical results that are obtained using 5 different human samples. Lane 1: genomic control. The 0.2 kb band is the Alu1 repeat PCR product, while the 6 kb band is the product of L1 repeat PCR. The total amount of genomic template is derived from the intensity of the 0.2 kb band while the 6 kb (L1)/0.2 kb (Alu1) ratio provides a qualitative measure of the template. Using a normalized L1/Alu1 ratio from Lane 1 the following assessments can be made of the five samples. Lane 2: sample contains 2×DNA but has a quality metric (L1/Alu1 sample)/(L1/Alu1 control) of <0.25. Lane 3: sample has 0.25×DNA and a very low quality metric (about 0). Lane 4: sample has 2×DNA and a quality metric of 1. Lane 5 sample has 2×DNA but a quality metric of <0.2 with an extensive degradation of the L1 repeat (see multiple bands). Lane 6 sample has 0.25×DNA with a quality metric of about 1. Thus, from these analyses the most suitable samples for aCGH assays are sample nos. 4 and 6 providing the sample concentrations are adjusted according to the results obtained.

The preceding merely illustrates principles of exemplary embodiments of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of evaluating a genomic sample, comprising:
a) amplifying a relatively short nucleic acid sequence from said genomic sample to produce an amount of a relatively low molecular weight amplification product;
b) amplifying a relatively long nucleic acid sequence from said genomic sample to produce an amount of a relatively high molecular weight amplification product; and
c) comparing the amount of said relatively low molecular weight amplification product to said amount of said high relatively molecular weight amplification product, to evaluate said genomic sample.

2. The method of claim 1, wherein said comparing produces a ratio and wherein said method further comprises:
d) comparing said ratio to a reference ratio.

3. The method of claim 2, wherein said reference ratio is obtained using a control genomic sample.

4. The method of claim 1, wherein said relatively long nucleic acid sequence is at least 10-fold greater in size than said relatively short nucleic acid sequence.

5. The method of claim 1, wherein said relatively short nucleic acid sequence is no more than 500 bp in length and said relatively long nucleic acid sequence is at least 3 kb in length.

6. The method of claim 1, wherein said relatively nucleic acid genomic sequence is a first repetitive element.

7. The method of claim 6, wherein said first repetitive element is present in a genome of said genomic sample at a copy number of at least 10,000.

8. The method of claim 7, wherein said first repetitive element is an Alu repeat.

9. The method of claim 1, wherein said relatively long nucleic acid sequence is a second repetitive element.

10. The method of claim 9, wherein said second repetitive element is present in a genome of said genomic sample at a copy number of at least 10,000.

11. The method of claim 10, wherein second repetitive element is a LINE element.

12. The method of claim 1, wherein said relatively high molecular weight product is at least 10-fold greater in molecular weight than said relatively low molecular weight product.

13. The method of claim 1, wherein said relatively low molecular weight product is no more than 500 bp in length and said relatively high molecular weight product is at least 3 kb in length.

14. The method of claim 1, wherein said method comprises amplifying said relatively short and relatively long nucleic acid sequences by polymerase chain reaction using primers that specifically bind to said relatively short and relatively long nucleic acid sequences.

15. The method of claim 1, wherein said assessing steps a) and b) are qualitative.

16. The method of claim 1, wherein said assessing steps a) and b) are quantitative.

17. The method of claim 1, wherein said method comprises separating said low molecular weight amplification product and said high molecular weight amplification product on the basis of their size.

18. The method of claim 1, wherein said genomic sample is made from a stored cellular sample.

19. A method of assessing integrity of a test genomic sample, comprising:
performing the method of claim 1 on said test genomic sample to produce a ratio; and
comparing said ratio to a reference ratio;
to produce an assessment of the integrity of said test genomic sample.

20. A method of identifying a test genomic sample suitable for use, comprising:
performing the method of claim 19 on said test genomic sample to produce an assessment of the integrity of said test genomic sample; and
determining whether said assessment is above a threshold;
wherein a test genomic sample having an assessment above said threshold indicates that said test genomic sample is suitable for use.

21. The method of claim 20, wherein said threshold is arbitrarily selected.

22. The method of claim 20, wherein an assessment above said threshold indicates that said genomic sample is suitable for use in an array-based comparative genome hybridization assay.

23. The method of claim 20, wherein an assessment above said threshold indicates that said genomic sample is suitable for amplification.

24. A method of selecting a test genomic sample, comprising:
performing the method of claim 20 on a plurality of test genomic samples; and
selecting a test genomic sample from said plurality of test genomic samples based on whether said numerical assessment is above said threshold.

25. A method comprising:
identifying a test genomic sample suitable for use in an array-based comparative genome hybridization assay using the method of claim 20; and
employing said test genomic sample in an array-based comparative genome hybridization assay.

26. The method of claim 25, wherein said employing step comprises:
labeling said test genomic sample to produce a labeled sample;
contacting said labeled sample with an polynucleotide array; and
detecting the presence of binding complexes on the surface of said array to assay said sample.

27. A kit comprising:
a first primer pair for amplifying a relatively short genomic sequence from a test genomic sample to produce a relatively low molecular weight amplification product;
a second primer pair for amplifying a relatively long genomic sequence from a test genomic sample to produce a relatively high molecular weight amplification product.

28. The kit of claim 27, further comprising a control genomic sample having an intact genome.

29. The kit of claim 27, further comprising reagents for labeling said genomic sample.

30. The kit of claim 27, further comprising a CGH array.

31. The kit of claim 27, further comprising a size separation device for separating said low and high molecular weight amplificatoin products.

32. The kit of claim 27, further comprising instructions to perform the method of claim 1.
